(19) United States
(12) Patent Application Publication
(10) Pub. No.: US 2026/0272539 A1
(43) Pub. Date: Sep. 17, 2026

(54) HIGH-FREQUENCY ABLATION MEDICAL DEVICE FOR PANCREATIC CYSTIC LESION, HAVING SELF-EXPANDING SPHERICAL ELECTRODE EMBEDDED THEREIN

(71) Applicants: THE ASAN FOUNDATION, Seoul (KR); UNIVERSITY OF ULSAN FOUNDATION FOR INDUSTRY COOPERATION, Ulsan (KR)

(72) Inventors: Jung-Hoon PARK, Hanam-si (KR); Sang-Soo LEE, Seoul (KR); Yubeen PARK, Seoul (KR); Dongsung WON, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 19/168,695

(22) PCT Filed: Dec. 18, 2023
(86) PCT No.: PCT/KR2023/020865

§ 371 Date: Sep. 24, 2025

(30) Foreign Application Priority Data
Mar. 27, 2023 (KR) 10-2023-0039729

(51) Int. Cl. A61B 18/14; A61B 17/00; A61B 18/00

(52) U.S. Cl. CPC A61B 18/1492; A61B 2017/00867; A61B 2018/00482; A61B 2018/00577; A61B 2018/1435; A61B 2018/1475; A61B 2218/007

(57) ABSTRACT

A radiofrequency ablation medical device includes: a guide catheter movable to a lesion site of an organ; and an electrode catheter disposed in the guide catheter to be inserted through and protrude out from an end of the guide catheter, and including a shape memory electrode for ablation of the lesion site, wherein the guide catheter includes: an outer tube; an inner tube having an outer surface spaced apart from an inner surface of the outer tube, and a catheter passage through which the electrode catheter is movable; and a suction channel formed between the outer tube and the inner tube to aspirate lesion fluid generated at the lesion site.

### TECHNICAL FIELD

This study is related to the Development of a Self-Expandable Electrode Catheter System for Local Thermal Therapy for Malignant Biliary Strictures (Project Unique Number: 1711196795, Project Number: RS-2023-00238092) of the Pan-Ministry Full-cycle Medical Device Research and Development Project, conducted at Asan Medical Center in Seoul, supported by the Pan-Ministry Full-cycle Medical Device Research and Development Project Group funded by the Korea government (Ministry of Science and ICT, Ministry of Trade, Industry and Energy; Ministry of Health and Welfare; and Ministry of Food and Drug Safety), and the Research on Stent-Based Irreversible Electroporation Hybrid Technique for the Treatment of Gastrointestinal Strictures (Project Unique Number: 1711186905, Project Number: 2022R1C1C1003205) of the New Researcher Support Program, conducted at Asan Medical Center in Seoul, supported by the National Research Foundation of Korea funded by the Korea government (Ministry of Science and ICT).

### BACKGROUND ART

In general, the treatment of cystic lesions of the pancreas is performed by surgical resection when the lesion is judged to cause symptoms or has a risk of malignancy; otherwise, patients are monitored periodically through follow-up observations. Despite advances in surgical techniques, the postoperative morbidity rate remains relatively high, reported to be as much as 15% to 30%, and it is difficult to accurately determine the risk of malignancy with only imaging findings and tissue specimens obtained by endoscopic ultrasonography, so this treatment method may be insufficient for managing various pancreatic cystic lesions. In addition, as the understanding of pancreatic cystic lesions deepens, the criteria for surgical indications have become increasingly stringent. Therefore, there is a need to develop treatment methods that are relatively safe, minimally invasive, and yet effective.

In line with these changes, endoscopic ultrasound-guided radiofrequency ablation (EUS-RFA) has recently been employed for minimally invasive endoscopic ultrasound-based treatment. Radiofrequency ablation is a technique that treats lesions using a heat-generating probe, and EUS-RFA is performed by directly accessing pancreatic and peripancreatic lesions through the walls of the stomach and duodenum.

In this method, a fine needle-shaped electrode is used to access the pancreas, and a thin micro-needle electrode is required for treatment of regions such as the uncinate process or the head of the pancreas. While the fine needle-shaped electrode is advantageous for access, it requires repeated procedures in a fan-shaped manner with different directions after reaching the lesion, and in the case of the micro-needle, its slender structure may limit the feasibility of repeated ablations.

### Prior Art Document

(Patent Document 1) Korean Patent Application Publication No. 10-2021-0079083 (Published on Jun. 29, 2021)

### DETAILED DESCRIPTION OF INVENTION

### Technical Problems

Embodiments of the present disclosure have been devised in view of the aforementioned background, and are intended to provide a radiofrequency ablation medical device for pancreatic cystic lesions, equipped with a self-expandable round electrode that facilitates access to the lesion site and the ablation procedure.

### Technical Solution

A radiofrequency ablation medical device according to one embodiment of the present disclosure includes: a guide catheter movable to a lesion site of an organ; and an electrode catheter disposed in the guide catheter to be inserted through and to protrude out from an end of the guide catheter, and including a shape memory electrode for ablation of the lesion site, wherein the guide catheter includes: an outer tube; an inner tube having an outer surface spaced apart from an inner surface of the outer tube, and a catheter passage through which the electrode catheter is movable; and a suction channel formed between the outer tube and the inner tube to aspirate lesion fluid generated at the lesion site

Further, the guide catheter may further include a support member arranged in the suction channel to support the outer surface of the inner tube and the inner surface of the outer tube.

Further, the guide catheter may further include a handle having an end to which the outer tube and the inner tube are connected and a passage through which the electrode catheter passes.

Further, the handle may include: a handle body part including a handle passage through which the electrode catheter is movable; a handle suction part formed to be in communication with the suction channel to provide suction force to the suction channel; and a handle fixer part for securing the electrode catheter.

Further, the end of the guide catheter may have a fine needle shape that is formed to be inclined in a longitudinal direction.

Further, the electrode catheter may include: a moving rod configured to move along the catheter passage; the shape memory electrode, disposed at one end of the moving rod and configured to retain a shape corresponding to an expanded state thereof, and a catheter pusher connected to the other end of the moving rod.

Further, when expanded, the shape memory electrode may provide a mesh structure having a shape corresponding to a shape of the lesion site to be in close contact with the lesion site.

Further, the shape memory electrode may protrude out from the guide catheter when the catheter pusher is moved relative to the guide catheter in a direction approaching the guide catheter, and be retracted into the guide catheter when the catheter pusher is moved relative to the guide catheter in a direction away from the guide catheter.

### Effect of Invention

According to embodiments of the present disclosure, by providing the spherical, self-expandable shape-memory electrode inside the fine needle-shaped catheter, it is possible to improve accessibility to the lesion site and the ablation procedure.

Further, according to embodiments of the present disclosure, the end of the guide catheter is formed in a fine needle shape and the electrode catheter having a shape memory electrode is disposed inside the guide catheter, which enables radiofrequency ablation at the lesion site to be performed with ease.

Furthermore, according to embodiments of the present disclosure, lesion fluid (e.g., cyst fluid) within the lesion site can be aspirated through the fine needle-shaped end of the guide catheter, which reduces the risk of complications such as seeding, and enables precise targeting of the lesion.

In addition, according to embodiments of the present disclosure, by placing the shape memory electrode at the fine needle-shaped end of the guide catheter, the shape memory electrode can self-expand into a round shape after the lesion fluid is aspirated from the lesion site, which enables treatment of the lesion without repeated procedures.

### BRIEF DESCRIPTION OF THE DRAWINGS

FIG. 1 is a configuration diagram illustrating a radiofrequency ablation medical device according to one embodiment of the present disclosure.

FIG. 2 is a state diagram illustrating a state in which an electrode catheter is being withdrawn, in the radiofrequency ablation medical device according to one embodiment of the present disclosure.

FIG. 3 is a cross-sectional view taken along line “III-III”′ of FIG. 1.

FIG. 4 is a cross-sectional view taken along line “IV-IV” of FIG. 1.

FIG. 5 is a state diagram illustrating a state in which the radiofrequency ablation medical device according to one embodiment of the present disclosure is guided to a lesion site by an endoscopic device.

FIG. 6 is a state diagram illustrating a state in which a guide catheter of the radiofrequency ablation medical device according to one embodiment of the present disclosure enters a lesion site and aspirates lesion fluid from the lesion site.

FIG. 7 is a state diagram illustrating a state in which the electrode catheter of the radiofrequency ablation medical device according to one embodiment of the present disclosure is deployed and expanded at the lesion site.

FIG. 8 is a state diagram illustrating a state in which the electrode catheter of the radiofrequency ablation medical device according to one embodiment of the disclosure is ablating the lesion site.

### BEST MODE FOR CARRYING OUT THE INVENTION

Hereinafter, a preferred embodiment of the present disclosure for implementing the spirit of the present disclosure will be described in more detail with reference to the accompanying drawings.

However, in describing the present disclosure, detailed descriptions of known configurations or functions may be omitted to clarify the present disclosure.

When an element is referred to as being ‘connected’ to, ‘supported’ by, ‘accessed’ to, ‘supplied’ to, ‘transferred’ to, or ‘contacted’ with another element, it should be understood that the element may be directly connected to, supported by, accessed to, supplied to, transferred to, or contacted with another element, but that other elements may exist in the middle.

The terms used in the present disclosure are only used for describing specific embodiments, and are not intended to limit the present disclosure. Singular expressions include plural expressions unless the context clearly indicates otherwise.

Further, in the present disclosure, it is to be noted that expressions, such as the upper side, the lower side, and the side surface, are described based on the illustration of drawings, but may be modified if directions of corresponding objects are changed. For the same reasons, some components are exaggerated, omitted, or schematically illustrated in the accompanying drawings, and the size of each component does not fully reflect the actual size.

Terms including ordinal numbers, such as first and second, may be used for describing various elements, but the corresponding elements are not limited by these terms. These terms are only used for the purpose of distinguishing one element from another element.

In the present specification, it is to be understood that the terms such as “including” are intended to indicate the existence of the certain features, areas, integers, steps, actions, elements, combinations, and/or groups thereof disclosed in the specification, and are not intended to preclude the possibility that one or more other certain features, areas, integers, steps, actions, elements, combinations, and/or groups thereof may exist or may be added.

Hereinafter, a specific configuration of a radiofrequency ablation medical device according to one embodiment of the present disclosure will be described.

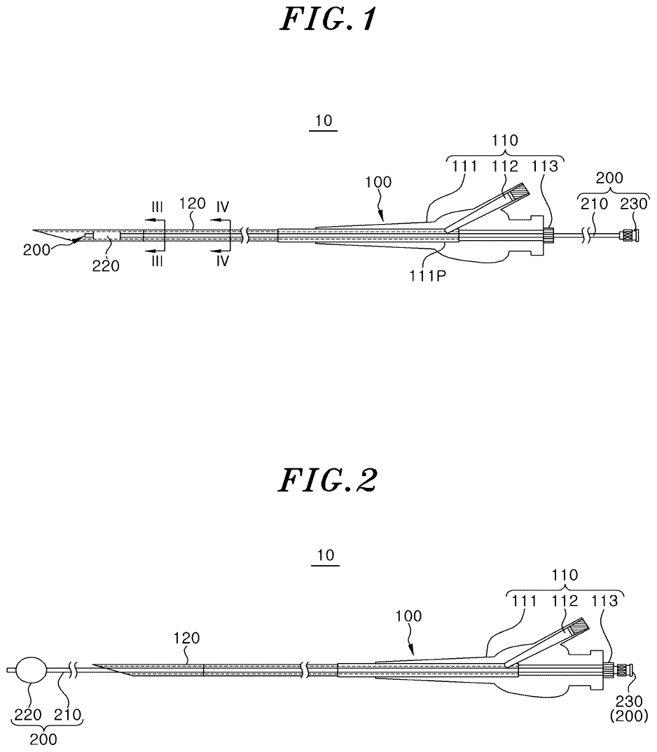

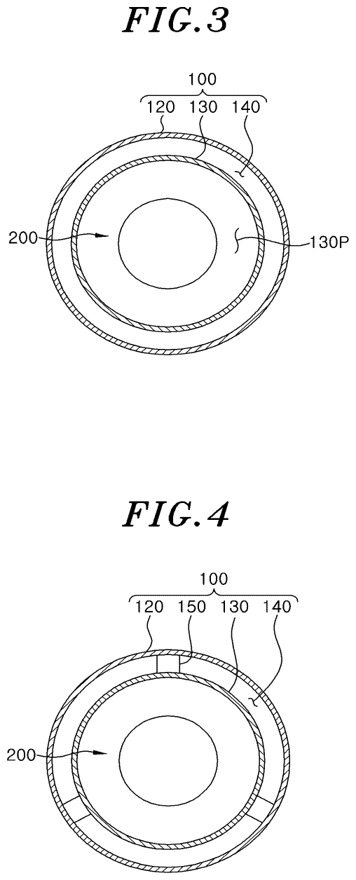

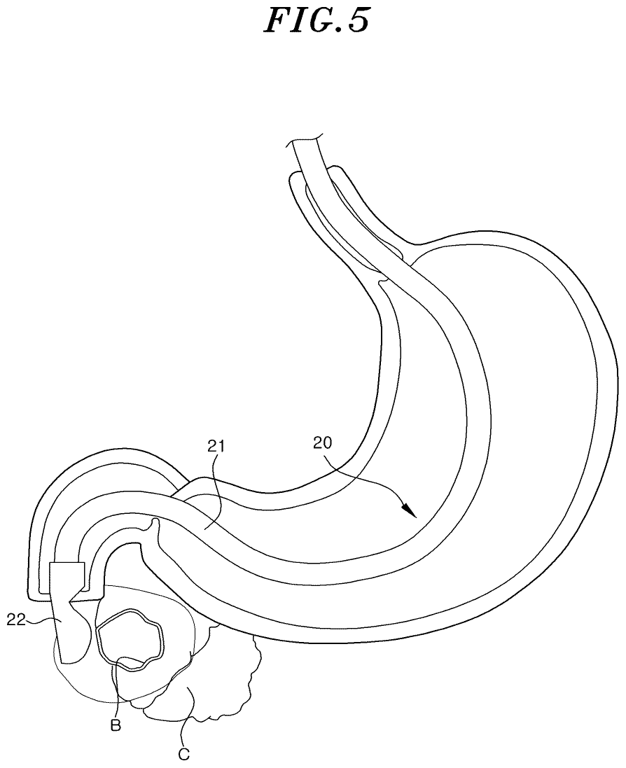

Referring to FIGS. 1 to 4, a radiofrequency ablation medical device 10 according to one embodiment of the present disclosure can facilitate radiofrequency ablation at a lesion site by using a fine needle-shaped end. The radiofrequency ablation medical device 10 may include a guide catheter 100 and an electrode catheter 200.

Specifically, the guide catheter 100 may be provided in the form of a probe that can be moved toward a lesion site of an organ. The guide catheter 100 can access the lesion site under the guidance of endoscopic ultrasound. As an example, the guide catheter 100 can access a cystic lesion site of the pancreas under the guidance of endoscopic ultrasound.

The end of the guide catheter 100 may be formed in a fine needle shape that is inclined in a longitudinal direction. In the present embodiment, the end of the guide catheter 100 is formed in a fine needle shape, but without being limited thereto, the end of the guide catheter 100 may be shaped in various pointed forms that allow easy access to the lesion site. The guide catheter 100 may include a handle 110, an outer tube 120, an inner tube 130, a suction channel 140, and a support member 150.

The handle 110 may be provided in the form of a grip that can be held by a user. An outer tube 120 and an inner tube 130 may be connected to an end of the handle 110. A passage through which the electrode catheter 200 can pass may be formed inside the handle 110. The handle 110 may include a handle body part 111, a handle suction part 112, and a handle fixer part 113.

The handle body part 111 may provide a handle passage 111P through which the electrode catheter 200 can move. The handle passage 111P may be formed to penetrate the handle body part 111. The handle body part 111 may be formed in a grip shape that is easy for a user to hold. The handle suction part 112 may be formed to be inclined at an upper portion of the handle body part 111, and the handle fixer part 113 may be disposed at a rear end of the handle body part 111.

The handle suction part 112 may be formed to be in communication with the suction channel 140. When negative pressure is applied to the handle suction part 112, the handle suction part 112 may provide suction force for aspirating lesion fluid from the lesion site to the suction channel 140. When suction force is applied through the handle suction part 112, the lesion fluid may be discharged to the outside through the suction channel 140 and the handle suction part 112.

The handle fixer part 113 can provide a locking function for securing the electrode catheter 200. The handle fixer part 113 may be rotatably disposed at the rear end of the handle body part 111. When the handle fixer part 113 is rotated, the handle fixer part 113 can selectively secure the electrode catheter 200. For example, when the handle fixer part 113 is rotated in one direction relative to the rear end of the handle body part 111, the handle fixer part 113 can be brought into close contact with to the electrode catheter 200 to lock it in place, and when the handle fixer part 113 is rotated in the other direction relative to the rear end of the handle body part 111, the handle fixer part 113 can release the fixation of the electrode catheter 200.

The outer tube 120 may be provided in the form of a tube that can entirely surround the inner tube 130. The inner tube 130 may be inserted and withdrawn through a front end of the outer tube 120, and a rear end of the outer tube 120 may be connected to the handle 110. The outer tube 120 may have an inner diameter larger than an outer diameter of the inner tube 130. A distance between an inner surface of the outer tube 120 and an outer surface of the inner tube 130 may be smaller than a distance between an inner surface of the inner tube 130 and an outer surface of a moving rod 210. When the distance between the inner surface of the outer tube 120 and the outer surface of the inner tube 130 is smaller than the distance between the inner surface of the inner tube 130 and the outer surface of the moving rod 210, the suction channel 140 can effectively aspirate lesion fluid from the lesion site even with a small suction force applied through the handle 110.

The inner tube 130 may be provided in the form of a tube that can entirely surround the electrode catheter 200. The inner tube 130 may provide a catheter passage 130P through which the electrode catheter 200 is movable. The inner tube 130 may have an inner diameter larger than an outer diameter of the electrode catheter 200. The inner tube 130 may be disposed inside the outer tube 120 with a spacing therebetween to define the suction channel 140. The inner tube 130 may have the outer diameter smaller than the inner diameter of the outer tube 120. In addition, a thickness of the inner tube 130 may be the same as or thicker than a thickness of the outer tube 120.

The suction channel 140 may be formed between the outer tube 120 and the inner tube 130. In the suction channel 140, the lesion fluid generated at the lesion site can be moved. One end of the suction channel 140 can aspirate the lesion fluid generated at the lesion site when suction force is applied to the other end of the suction channel 140 and negative pressure is generated within the suction channel 140.

In the present embodiment, the longitudinal cross-sectional area of the suction channel 140 is formed in the same shape along the extension direction of the guide catheter 100, but without being limited thereto, the longitudinal cross-sectional area of the suction channel 140 may be formed in different shapes along the extension direction of the guide catheter 100. For example, the longitudinal cross-section of the suction channel 140 located at a front end (one end) of the guide catheter 100 may be larger than the longitudinal cross-section of the suction channel 140 located at a rear end (the other end) of the guide catheter 100.

The support member 150 may be arranged between the inner tube 130 and the outer tube 120 so that the suction channel 140 is formed. The support member 150 may support the outer surface of the inner tube 130 and the inner surface of the outer tube 120 within the suction channel 140. A width of the support member 150 may correspond to the spacing distance between the inner tube 130 and the outer tube 120. The support members 150 may be provided as a plurality of support members 150 disposed to be spaced apart from each other in the circumferential direction between the inner tube 130 and the outer tube 120. The plurality of support members 150 may also be spaced apart from each other in the extension direction of the guide catheter 100 between the inner tube 130 and the outer tube 120. For example, three support member 150 may be disposed to be spaced apart from each other between the inner tube 130 and the outer tube 120. The three support members 150 may also be spaced apart in the extension direction of the guide catheter 100. The width of the support member 150, that is, the length of the support member 150 extending in the circumferential direction of the outer tube 120, may be shorter than the distance between the inner surface of the outer tube 120 and the outer surface of the inner tube 130. When the width of the support member 150 is shorter than the distance between the inner surface of the outer tube 120 and the outer surface of the inner tube 130, the resistance of the support member 150 to the movement of the lesion fluid through the suction channel 140 can be minimized.

When the electrode catheter 200 is positioned at a lesion site of an organ, the electrode catheter 200 can perform thermal ablation on the lesion site through a shape memory electrode 220. The electrode catheter 200 may be movably disposed inside the guide catheter 100. When the rear end of the electrode catheter 200 is pushed from the handle 110, the leading end of the electrode catheter 200 can protrude from the end of the guide catheter 100, and when the rear end of the electrode catheter 200 is pulled from the handle 110, the leading end of the electrode catheter 200 can be retracted into the end of the guide catheter 100. The electrode catheter 200 may include a moving rod 210, a shape memory electrode 220, and a catheter pusher 230.

The moving rod 210 may be made of a flexible material that can be moved along the catheter passage 130P of the guide catheter 100. The shape memory electrode 220 may be disposed at a leading end of the moving rod 210, and the catheter pusher 230 may be connected to a rear end of the moving rod 210. When the catheter pusher 230 is pushed or pulled from the handle 110, the moving rod 210 may be moved forward or backward along the catheter passage 130P of the guide catheter 100.

The shape memory electrode 220 may be fixed to an outer surface of the moving rod 210 at the front end of the moving rod 210. When power is supplied from a separate energy supply device, the shape memory electrode 220 can perform thermal ablation on a lesion of an organ using thermal energy (radiofrequency energy).

The shape memory electrode 220 may be made of a shape memory material capable of restoring the shape of its expanded shape. When expanded, the shape memory electrode 220 may provide a mesh structure having a shape corresponding to the shape of the lesion site, allowing close contact with the lesion site. For example, the shape memory electrode 220 may be a unitary nitinol wire with shape-memory properties. As a matter of course, the shape memory electrode 220 may be composed of various types of shape memory materials capable of restoring the shape of the expanded state in addition to the nitinol wire. Further, when contracted, the shape memory electrode 220 can be retracted into the guide catheter 100 while wrapping around the outer surface of the moving rod 210.

When expanded, the shape memory electrode 220 may have a shape corresponding to the lesion site of an organ, allowing it to closely confirm to and contact the lesion site. For example, the shape memory electrode 220 may have a spring shape that is twisted into a spiral shape to have an outer diameter corresponding to the inner diameter of the lesion site. In a state where the shape memory electrode 220 is in contact with the lesion site, the shape memory electrode 220 can perform radiofrequency thermal treatment on the lesion site by applying thermal energy to the lesion site.

The catheter pusher 230 may be provided in the form of a handle for moving the shape memory electrode 220. The catheter pusher 230 may be connected to the other end of the moving rod 210. When the catheter pusher 230 is moved relative to the guide catheter 100 in a direction approaching the guide catheter 100, the shape memory electrode 220 may be deployed from the guide catheter 100. When the catheter pusher 230 is moved relative to the guide catheter 100 in a direction away from the guide catheter 100, the shape memory electrode 220 may be retracted into the guide catheter 100 for retrieval.

Hereinafter, the operation and effects of a radiofrequency ablation medical device having the configuration described above will be described.

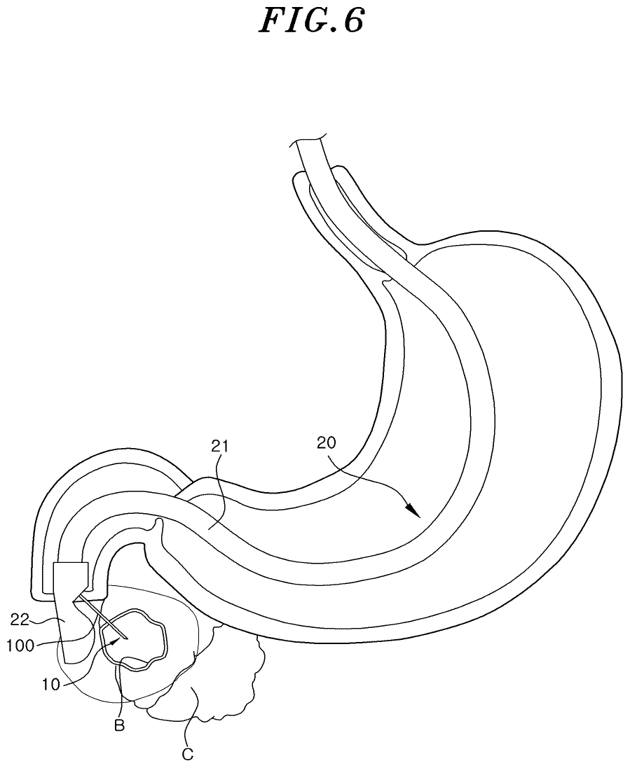

Referring to FIG. 5, in order to guide the radiofrequency ablation medical device 10 to a lesion site B of an organ C having a malignant stricture, first, the radiofrequency ablation medical device 10 may be moved to the lesion site B through an endoscopic channel part 21 of an endoscopic device 20. In the present embodiment, the radiofrequency ablation medical device 10 can be moved to the lesion site B through the endoscopic channel part 21 under the guidance of an endoscopic ultrasound part 22.

Referring to FIG. 6, when the endoscopic ultrasound part 22 of the endoscopic device 20 is moved to the lesion site B, the end of the guide catheter 100, having a fine needle shape, can be advanced to the lesion site B. When the end of the guide catheter 100 is positioned at the lesion site B of the pancreas C, the radiofrequency ablation medical device 10 can aspirate the lesion fluid generated at the lesion site through the guide catheter 100 by applying negative pressure to the suction channel 140.

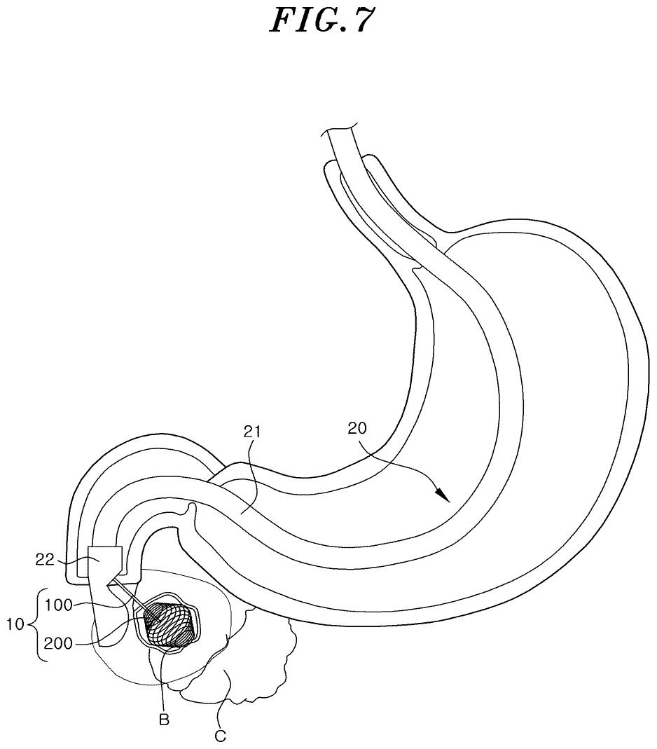

Referring to FIG. 7, when the lesion fluid generated at the lesion site is aspirated through the guide catheter 100, the electrode catheter 200 protrudes from the end of the guide catheter 100, so that the shape memory electrode 220 of the electrode catheter 200 can expand at the lesion site B. The shape memory electrode 220 can be deployed and maintained at the lesion site B in a shape corresponding to the shape of the lesion site, which is memorized.

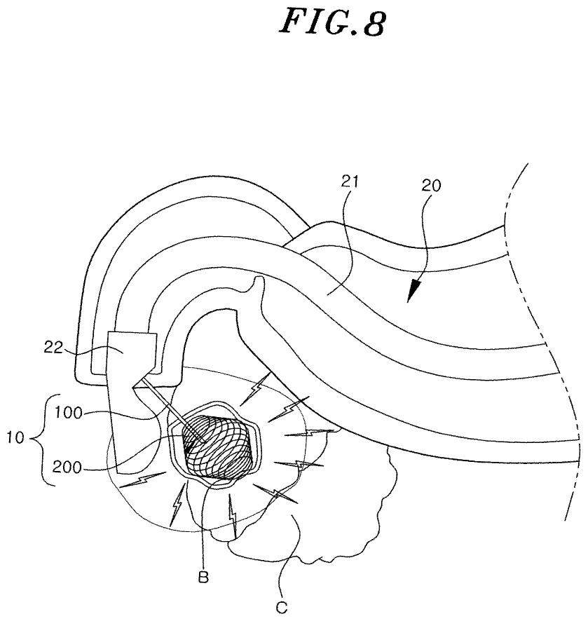

Referring to FIG. 8, in a state where the shape memory electrode 220 is expanded and in close contact with the lesion site B, when power is supplied to the shape memory electrode 220, the shape memory electrode 220 supplied with power applies thermal energy to the lesion site B, thereby performing radiofrequency thermal treatment (radiofrequency ablation) on the lesion site B. Thereafter, when the radiofrequency thermal treatment on the lesion site B is completed, the shape memory electrode 220 can be retracted into the end of the catheter 100 of the radiofrequency ablation medical device 10, and the radiofrequency ablation medical device 10 with the retracted shape memory electrode 220 can be removed from the lesion site B and moved to its initial position.

As described above, according to the present disclosure, the shape memory electrode capable of self-expanding in a spherical shape is provided inside the fine needle-shaped catheter, thereby improving accessibility and ease of treatment for a lesion site. The end of the guide catheter is formed in a fine needle shape, and the electrode catheter with the shape memory electrode is disposed inside the guide catheter, so that radiofrequency ablation of the electrode catheter for the lesion site can be easily performed. Lesion fluid within the lesion site can be aspirated, which reduces the risk of complications such as seeding, and enables precise targeting of the lesion, and repeated ablation is not required, resulting in reduced procedure time.

The examples of the present disclosure have been described above as specific embodiments, but these are only examples, and the present disclosure is not limited thereto, and should be construed as having the widest scope according to the technical spirit disclosed in the present specification. A person skilled in the art may combine/substitute the disclosed embodiments to implement a pattern of a shape that is not disclosed, but it also does not depart from the scope of the present disclosure. In addition, those skilled in the art can easily change or modify the disclosed embodiments based on the present specification, and it is clear that such changes or modifications also belong to the scope of the present disclosure.

1. A radiofrequency ablation medical device comprising:
a guide catheter movable to a lesion site of an organ, and
an electrode catheter disposed in the guide catheter to be inserted through and to protrude out from an end of the guide catheter, and including a shape memory electrode for ablation of the lesion site,
wherein the guide catheter includes:
an outer tube;
an inner tube having an outer surface spaced apart from an inner surface of the outer tube, and a catheter passage through which the electrode catheter is movable; and
a suction channel formed between the outer tube and the inner tube to aspirate lesion fluid generated at the lesion site.

2. The radiofrequency ablation medical device of claim 1, wherein the guide catheter further includes a support member arranged in the suction channel to support the outer surface of the inner tube and the inner surface of the outer tube.

3. The radiofrequency ablation medical device of claim 1, wherein the guide catheter further includes a handle having an end to which the outer tube and the inner tube are connected and a passage through which the electrode catheter passes.

4. The radiofrequency ablation medical device of claim 3, wherein the handle includes:
a handle body part including a handle passage through which the electrode catheter is movable;
a handle suction part formed to be in communication with the suction channel to provide suction force to the suction channel; and
a handle fixer part for securing the electrode catheter.

5. The radiofrequency ablation medical device of claim 1, wherein the end of the guide catheter has a fine needle shape that is formed to be inclined in a longitudinal direction.

6. The radiofrequency ablation medical device of claim 1, wherein the electrode catheter includes:
a moving rod configured to move along the catheter passage;
the shape memory electrode, disposed at one end of the moving rod and configured to retain a shape corresponding to an expanded state thereof; and
a catheter pusher connected to the other end of the moving rod.

7. The radiofrequency ablation medical device of claim 6, wherein when expanded, the shape memory electrode provides a mesh structure having a shape corresponding to a shape of the lesion site to be in close contact with the lesion site.

8. The radiofrequency ablation medical device of claim 6, wherein the shape memory electrode protrudes out from the guide catheter when the catheter pusher is moved relative to the guide catheter in a direction approaching the guide catheter, and is retracted into the guide catheter when the catheter pusher is moved relative to the guide catheter in a direction away from the guide catheter.
